(19) United States
(12) Patent Application Publication
(10) Pub. No.: US 2015/0313562 A1
(43) Pub. Date: Nov. 5, 2015

(54) METHOD FOR RETRIEVING APPLICATION COMMANDS, COMPUTING UNIT AND MEDICAL IMAGING SYSTEM

(71) Applicant: Siemens Aktiengesellschaft, Muenchen (DE)

(72) Inventor: Sören KUHRT, Erlangen (DE)

(21) Appl. No.: 14/689,350

(22) Filed: Apr. 17, 2015

(30) Foreign Application Priority Data
Apr. 30, 2014 (DE) 102014208222.6

(51) Int. Cl. A61B 6/00; A61B 6/03; G06F 19/00; G06F 3/0488

(52) U.S. Cl. CPC A61B 6/467; G06F 3/04883; A61B 6/032; G06F 19/3406; A61B 6/461; A61B 6/548

(57) ABSTRACT

A method is disclosed for retrieving application commands relating to a medical image displayed on a touch-capable user interface of a computing unit of a medical imaging system. In at least one embodiment, the method includes at least detecting simultaneous touching of the user interface on at least two touch points and displaying the application-situation-related possible application commands on the user interface at the touch points. A computing unit of a medical imaging system is also disclosed, including at least one memory unit and one program code stored in the memory unit, wherein the program code is embodied such that a medical image of a patient is output on a touch-capable user interface of the computing unit, wherein the method of an embodiment of the invention can be executed. Finally, a medical imaging system is disclosed, including at least one computing unit according to an embodiment of the invention.

### PRIORITY STATEMENT

The present application hereby claims priority under 35 U.S.C. §119 to German patent application number DE 102014208222.6 filed Apr. 30, 2014, the entire contents of which are hereby incorporated herein by reference.

### FIELD

At least one embodiment of the invention generally relates to a method for retrieving application commands relating to a medical image displayed on a touch-capable user interface of a computing unit of a medical imaging system. At least one embodiment of the invention further generally relates to a computing unit of a medical imaging system and a medical imaging system.

### BACKGROUND

Touch-screen monitors or touch-capable user interfaces are known. These provide suitable input media for operating software in many situations and are above all used where the use of a keyboard and mouse is not convenient, for example when entries have to be made while standing. A specific example of this would be the use of a touch-screen monitor attached to the gantry of a CT system, wherein the software is then exclusively operated via the touch monitor.

Here, the possible applications are to be displayed on the touch-screen monitor such that, on the one hand, inexperienced users can find the desired application without assistance and experienced users can reach the most commonly used applications quickly and with no time loss. The user should also be able to extend and improve his experiences and knowledge without any explicit learning effort.

To date, three different input and display variants are mainly used, although each of these has certain disadvantages:

When using fixed menus or other interface elements, the available usage options are offered at certain, predefined positions. Inexperienced users usually only find the desired application by searching through the whole menu. Experienced users on the other hand are not really able to optimize their productivity with this method. To activate an application, it is necessary for the desired application command to be recognized and reached with the hand. For this, the hand with the mouse or on the touch-screen monitor has to be moved from the present position to the position of the next application thus requiring the user to have eye-hand coordination. With many applications, this step has to be repeated several times. This method of software control is above all impracticable if, for example, the user is standing while working.

Another possibility is the use of so-called context menus. In this case, the user is able to display an abbreviated menu field of the available applications at the position of the respective application by a prespecified gesture, for example by touching the touch-screen monitor for longer, the so-called “press and hold” command. The desired application can be selected by further contact. Inexperienced users can also easily find the desired application with this method, wherein only the touch command or the gesture for activating the context menu has to be learned. However, this also offers experienced users few opportunities for improvement. Although, there is virtually no longer any need for the hand to leave the present position, once again eye-hand coordination is necessary to select the desired application in the menu. Moreover, the “press and hold” command usually used costs unnecessary time.

As a further possibility, with many software products for frequently used applications, firmly defined gestures or commands are used, for example “swiping with two fingers from left to right”. Experienced users achieve high productivity with this method. However, inexperienced users cannot use this method since the existence and meaning of the available gestures is not obvious. To develop from an inexperienced user to an experienced user, it is necessary explicitly to consult documentation and to learn the possible gestures.

Another possibility is offered by the so-called Arpege method with which specific commands are input and applications are executed by successively touching the touch-screen monitor in special combinations with individual fingers and applications. The gestures are executed with a hand, wherein the individual fingers touch the touch-screen monitor in succession. The hand also has to be moved when changing the application and when inputting further commands. Therefore, once again, this method requires the user to have good eye-hand coordination.

### SUMMARY

At least one embodiment of the invention provides a simplified method for retrieving application commands on a touch-capable user interface.

Advantageous developments of the invention are the subject matter of subordinate claims.

At least one embodiment of ther invention is directed to a method for retrieving application-situation-related possible application commands relating to a medical image displayed on a touch-capable user interface of a computing unit of a medical imaging system, comprising:

detecting simultaneous touching of the user interface on at least two touch points; and

displaying application-situation-related possible application commands on the user interface at the at least two touch points.

The inventor also suggests, in at least one embodiment, improving a computing unit of a medical imaging system comprising at least one memory unit and one program code stored in the memory unit, wherein the program code is embodied such that a medical image of a patient is output on a touch-capable user interface of the computing unit so that the above-described method according to at least one embodiment of the invention method can be executed.

Finally, the inventor suggests a medical imaging system in at least one embodiment, in particular a CT system, comprising at least one above-described computing unit according to at least one embodiment of the invention. The medical imaging system is preferably a CT system. Alternatively, this is an MRT system.

### BRIEF DESCRIPTION OF THE DRAWINGS

The following describes the invention with reference to a preferred example embodiment with the aid of the drawing, wherein only the features required to understand the invention are illustrated.

The following reference symbols are used: 1: Touch-capable user interface; 2: Medical image; 3.1 to 3.4: Fingers; A1 to A8: Application commands; B1 to B4: Touch points; C1: CT system; C2: First X-ray tube; C3: First detector; C4: Second X-ray tube (optional); C5: Second detector (optional); C6: Gantry housing; C7: Patient; C8: Patient bed; C9: System axis; C10: Computing and control unit; Prg₁ to Prgₙ: Computer programs; S1 to S4: Steps of the method.

The individual figures show:

FIG. 1 a schematic image of a CT system with a control and computing unit,

FIG. 2 a touch-capable user interface with a medical image and possible application commands,

FIG. 3 the touch-capable user interface with the medical image shown in FIG. 2 and further possible application commands,

FIG. 4 a segment of the touch-capable user interface with the medical image shown in FIG. 2 with the fingers of a user's hand and

FIG. 5 a schematic image of the steps of the method according to an embodiment of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The inventor has recognized that a combination of context-dependent menus with a simple sequence of touch commands represents a simplified method for retrieving possible applications.

Here, the context-dependent menu can be activated, i.e. displayed, by simultaneously touching a touch-capable user interface at several touch points. For example, the user touches the user interface simultaneously with four fingers of one hand. On the user interface, that is for example at the touch points, the user is now shown the possible commands for the respective application situation. At the same time, an application command can be assigned to each touch point. If there are still further commands as touch points to be executed in this application situation, the user can cause these to be displayed by briefly tapping one of the touch points. For purposes of simplicity, the further commands can be displayed at the same touch points so that, once again, an application command is assigned to each touch point. Tapping on a command again enables the user to select and execute this command.

The eye-hand coordination required with an embodiment of this method is extremely low. The user does not have to move his hand from the position first adopted during the entry or menu navigation. There is only a short movement of the fingers when tapping on the commands on the touch points first selected on the user interface. In this case, the positions of the touch points are simply determined by the finger positions with a relaxed hand position.

The fact that the available application commands are offered and displayed at the original touch points makes quick selection of the desired commands possible. Inexperienced users only have to note the gesture for opening the menu. This is simple and requires virtually no learning effort. The further steps are self-explanatory and all available application commands can be reached without any additional prior knowledge on the part of the user.

Experienced users can also note the tap sequences of the most commonly used application commands. These sequences can then be executed without any eye-hand coordination in a very short time. During this, the hand does not leave its present position. Inexperienced users are able to learn the sequences without conscious effort or the assistance of documentation.

Accordingly, the inventor suggests a method, in at least one embodiment, for retrieving application commands relating to a medical image displayed on a touch-capable user interface of a computing unit of a medical imaging system comprising at least:

detecting simultaneous touching of the user interface on at least two touch points and displaying the application-situation-related possible application commands on the user interface at the touch points.

The method according to at least one embodiment of the invention is simple to learn and carry out. Only minimal hand-eye-coordination is required since the hand is not moved away from the first position adopted on the user interface. The simultaneous touching advantageously takes the form of briefly tapping or holding the touch points.

According to at least one embodiment of the invention, the application commands are displayed at the touch points. At the same time, an application command is advantageously assigned to each touch point. In a preferred application command, in each case in the immediate vicinity of a touch point, a short descriptor for the assigned application command is displayed, for example “Copy”, “Paste”, “More” etc.

In a further step of the method according to at least one embodiment of the invention, tapping on of a specific touch point, that is selecting a specific application command causes further application-situation-related possible application commands to be displayed. This step can be executed if an application situation has more possible commands than are simultaneously displayed at the touch points in the first step. Advantageously, then the command to open and display the further possible commands is displayed at one of the touch points, for example identified with the descriptor “Paste”.

For purposes of simplicity, the further application commands are preferably displayed at the same touch points as the commands already displayed in the first step. Hence, the user's hand can remain in its original position. Only the fingers of the hand are moved when tapping the user interface.

In order, in a further step, to select and execute an application command, advantageously the respective touch point assigned to this application command is tapped. The user can note the sequence of touching, i.e. tapping or holding and then tapping again, for the most common application commands, similarly to a piano player, and hence execute the method according to at least one embodiment of the invention intuitively.

A preferred embodiment of the method according to the invention provides that the user interface is touched simultaneously at four touch points. This reduces the situations in which there are more possible application commands than touch points are executed or application commands are displayed in the first step thus necessitating a further intermediate step to display the further application commands. The user interface can advantageously be touched with four fingers of a user's hand. Preferably, these are the index finger, middle finger, ring finger and little finger of a hand. At the same time, the positions of the touch points on the user interface are in principle freely selectable. Advantageously, the positions of the touch points are determined simply by anatomically conditioned finger positioning. This corresponds, for example, to a semi-circle-like arrangement of the touch points on the user interface.

The inventor also suggests, in at least one embodiment, improving a computing unit of a medical imaging system comprising at least one memory unit and one program code stored in the memory unit, wherein the program code is embodied such that a medical image of a patient is output on a touch-capable user interface of the computing unit so that the above-described method according to at least one embodiment of the invention method can be executed.

Finally, the inventor suggests a medical imaging system in at least one embodiment, in particular a CT system, comprising at least one above-described computing unit according to at least one embodiment of the invention. The medical imaging system is preferably a CT system. Alternatively, this is an MRT system.

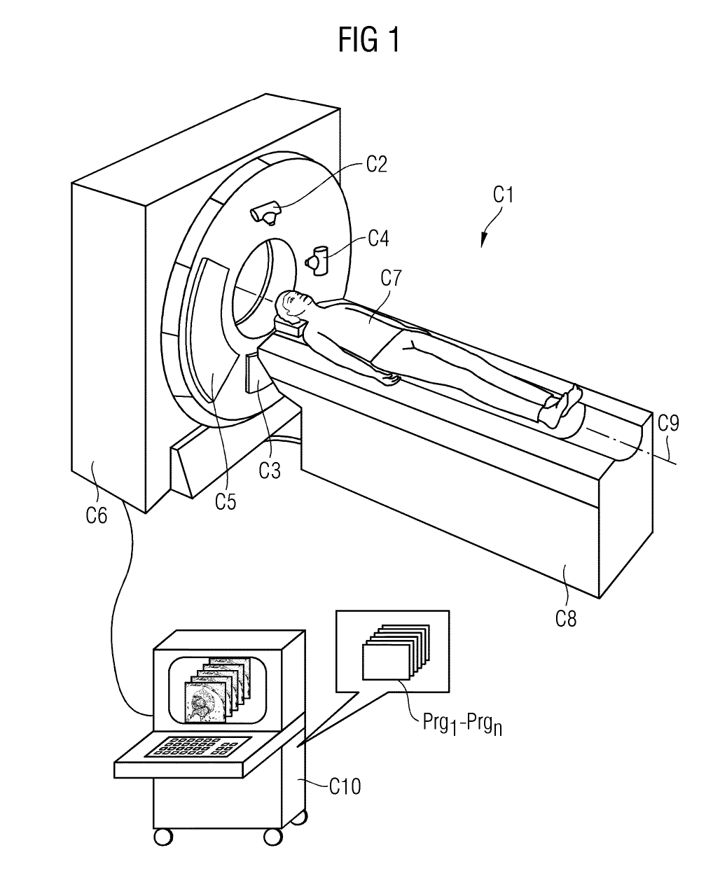

FIG. 1 shows an example CT system C1. The CT system C1 comprises a gantry housing C6 containing a gantry, which is not shown in any further detail here, to which a first X-ray tube C2 with an opposing first detector C3 is secured. Optionally, a second X-ray tube C4 with a second opposing detector C5 is provided. A patient C7 is positioned on a patient bed C8 that is displaceable in the direction of the system axis C9, with which the patient can be pushed during the scanning with the X-rays continuously or sequentially along the system axis C9 through a measuring field between the X-ray tubes C2 and C4 and the respectively assigned detectors C3 and C5. This process is controlled by a computing and control unit C10 with the aid of computer programs Prg₁ to Prgₙ.

The computing and control unit C10 enables the execution of the method according to an embodiment the invention for retrieving application commands relating to a medical image displayed on a touch-capable user interface of the computing and control unit C10.

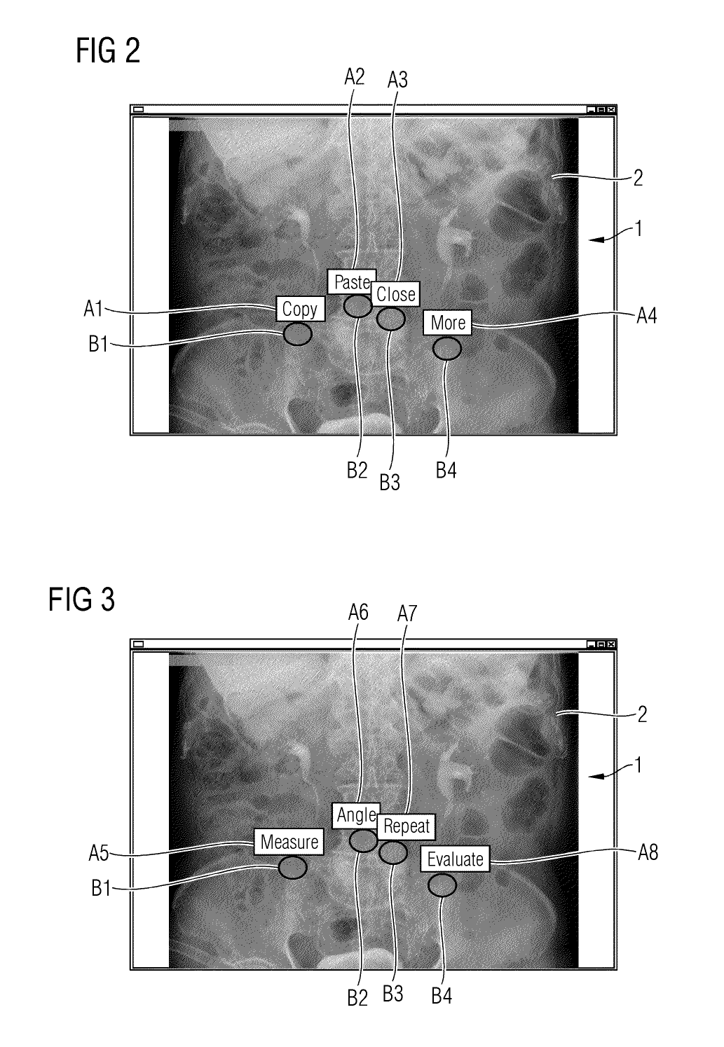

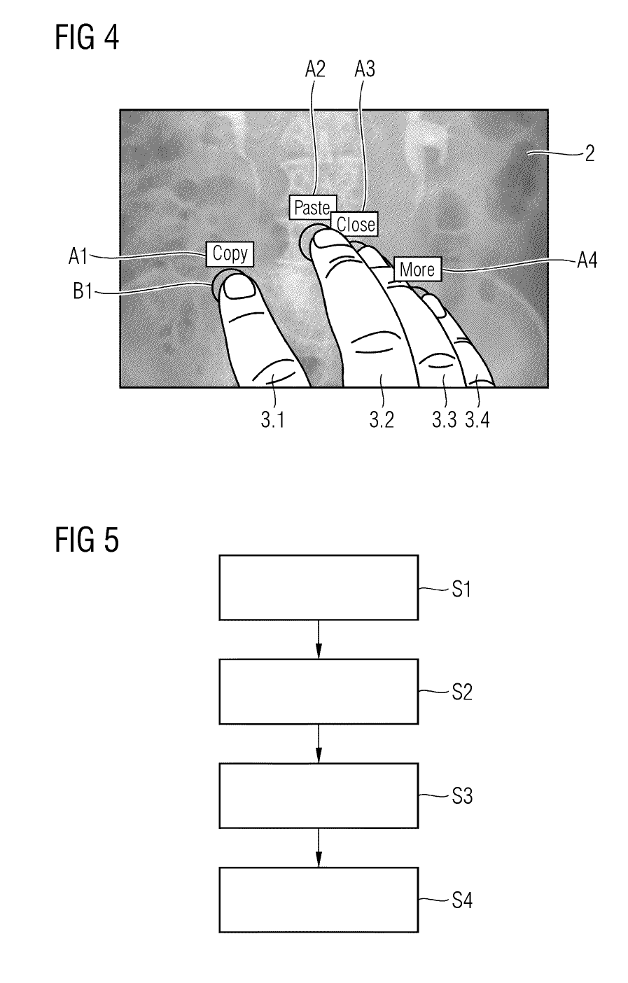

FIG. 2 shows a touch-capable user interface 1 with a medical image 2 and possible application commands A1 to A4. The medical image 2 shows an image taken with a CT system C1 of a human skeleton in the pelvic region. The user interface 1 shows four application commands A1 to A4, namely “Copy”, “Paste”, “Close” and “More”. In this case, the application commands A1 to A4 appear after the simultaneous touching of the touch-capable user interface 1 at four touch points B1 to B4, see also FIG. 4. Here, a short tap is sufficient for the touching. The application commands A1 to A3 are the most commonly used commands in the application situation shown here. Each application command A1 to A4 is arranged directly next to a touch point B1 to B4 and hence assigned thereto. Here, the application command A2 is assigned to the touch points B2.

Tapping the touch point B4, causes application command A4 “More” to be selected and executed. This command causes four further possible application commands A5 to A8 to be displayed at the same four touch points B1 to B4. The further application commands A5 to A8 are also each assigned to the touch points B1 to B4. This is depicted in FIG. 3. The further application commands A5 to A8 are “Measure” A5, “Angle” A6, “Repeat” A7 and “Evaluate” A8.

FIG. 4 shows a segment of the touch-capable user interface 1 with the medical image 2 in FIG. 2 with four fingers 3.1 to 3.4 of the right hand of a user. The four fingers 3.1 to 3.4 touch the user interface 1 simultaneously at the four touch points B1 to B4 at which the application commands A1 to A4 are displayed. Only one finger movement is needed for the further tapping and selection of an application command. The positions of touch points B1 to B4 are arranged in accordance with loose, anatomically conditioned hand or finger positioning in an approximate semicircle on the user interface 1.

In the example shown in FIGS. 2 and 3, the tapping sequence for executing the application command “Copy” would be: four-finger tap+index finger tap or to execute the application command “Repeat”: four-finger tap+small-finger tap+ring finger tap.

FIG. 5 shows a schematic image of the steps of the method according to an embodiment of the invention for retrieving application commands relating to a medical image displayed on a touch-capable user interface of a computing unit of a CT system. In a first step S1, simultaneous touching of the user interface at four touch points causes four application-situation-related possible application commands to be displayed, for example “Copy”, “Paste”, “Close” and “More”. In the application situation described here, the application command “More” is used to show that there are further possible application commands. If the user wishes to execute a command other than the commands displayed “Copy”, “Paste” or “Close”, he taps the application command “More” with the little finger in a further step S2, see also FIG. 4. Then four further application commands, for example “Measure”, “Angle”, “Repeat” and “Evaluate” are displayed at the same touch points of the first four application commands, step S3. Tapping one of the touch points causes the respective assigned application command to be selected and executed in a further step S4. When specific tapping sequences have been learned, the execution of the method according to the invention can be performed intuitively, in particular also without looking at the user interface.

Although the invention was illustrated and described in more detail by the preferred example embodiment, the invention is not restricted by the disclosed examples and other variations can be derived therefrom by the person skilled in the art without departing from the scope of protection of the invention.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for retrieving application-situation-related possible application commands relating to a medical image displayed on a touch-capable user interface of a computing unit of a medical imaging system, comprising:
detecting simultaneous touching of the user interface on at least two touch points; and
displaying application-situation-related possible application commands on the user interface at the at least two touch points.

2. The method of claim 1, further comprising:
detecting tapping of at least one of the at least two touch points, causing further application-situation-related possible commands to be displayed.

3. The method of claim 2, wherein the further application commands are displayed at the at least two touch points.

4. The method of claim 2, wherein the tapping of the touch point causes the respective application command to be executed.

5. The method of claim 1, wherein simultaneous touching of the user interface at four touch points is detected.

6. The method of claim 5, wherein the detecting detects the user interface being touched with fingers of a hand of a user.

7. The method of claim 1, wherein positions of the at least two touch points are freely selectable.

8. The method of claim 7, wherein the positions of the at least two touch points are arranged on the user interface in accordance with anatomically conditioned finger positioning.

9. A computing unit of a medical imaging system, comprising:
at least one memory unit; and
at least one program code stored in the memory unit, wherein the at least one program code is embodied such that a medical image of a patient is output on a touch-capable user interface of the computing unit, wherein upon execution of the at least one program code, at least the following is executed
detecting simultaneous touching of the user interface on at least two touch points; and
displaying application-situation-related possible application commands on the user interface at the at least two touch points.

10. A medical imaging system, comprising:
at least one computing unit, the at least one computing unit is the computing unit of claim 9.

11. The method of claim 3, wherein the tapping of the touch point causes the respective application command to be executed.

12. The method of claim 6, wherein the detecting detects the user interface being touched with an index finger, middle finger, ring finger and little finger.

13. The medical imaging system of claim 10, wherein the medical imaging system is a CT system.
